SEQUENCE LISTING (11 sequences)

SEQ ID NO: 1 - DNA, 20 bp, synthetic construct
  source 1..20: mol_type = other DNA; note = forward primer F1 for amplification of 16S rDNA
agagtttgatcctggctcag

SEQ ID NO: 2 - DNA, 18 bp, synthetic construct
  source 1..18: mol_type = other DNA; note = reverse primer R1 for amplification of 16S rDNA
ggttaccttgttacgact

SEQ ID NO: 3 - DNA, 1367 bp, Leuconostoc lactis
  source 1..1367: mol_type = genomic DNA; note = 16S rDNA sequence of isolate LEL168
aaggtgcttgcacctttcaagcgagtggcgaacgggtgagtaacacgtggataacctgcctcaaggctggggataacatttggaaacagatgctaataccgaataaaacttagtatcgcatgatacaaagttgaaaggcgctacggcgtcacctagagatgggtccgcggtgcattagttagttggtggggtaaaggcctaccaagacaatgatgcatagccgagttgagagactgatcggccacattgggactgagacacggcccaaactcctacgggaggctgcagtagggaatcttccacaatgggcgaaagcctgatggagcaacgccgcgtgtgtgatgaaggctttagggtcgtaaagcactgttgtatgggaagaaatgctagaatagggaatgattctagttcgacggtaccataccagaaagggacggctaaatacgtgccagcagccgcggtaatacgtatgtcccgagcgttatccggatttattgggcgtaaagcgagcgcagacggttgattaagtctgatgtgaaagcccggagctcaactccggaatggcattggaaactggttaacttgagtgttgtagaggtaagtggaactccatgtgtagcggtggaatgcgtagatatatggaagaacaccagtggcgaaggcggcttactggacaacaactgacgttgaggctcgaaagtgtgggtagcaaacaggattagataccctggtagtccacaccgtaaacgatgaatactaggtgttaggaggtttccgcctcttagtgccgaagctaacgcattaagtattccgcctggggagtacgaccgcaaggttgaaactcaaaggaattgacggggacccgcacaagcggtggagcatgtggtttaattcgaagcaacgcgaagaaccttaccaggtcttgacatcctttgaagcttctagagatagaagtgttctcttcggagacaaagtgacaggtggtgcatggtcgtcgtcagctcgtgtcgtgagatgttgggttaagtcccgcaacgagcgcaacccttattgttagttgccagcattcagttgggcactctagcgagactgccggtgacaaaccggaggaaggcggggacgacgtcagatcatcatgccccttatgacctgggctacacacgtgctacaatggcgtatacaacgagttgccaacccgcgagggtgagctaatctcttaaagtacgtctcagttcggactgcagtctgcaactcgactgcacgaagtcggaatcgctagtaatcgcggatcagcacgccgcggtgaatacgttcccgggtcttgtacacaccgcccgtcacaccatgggagtttgtaatgcccaaag

SEQ ID NO: 4 - DNA, 33 bp, synthetic construct
  source 1..33: mol_type = other DNA; note = forward primer F2 for amplification of dnaA
ggtggcgttggtctaggwaaaacmcayytratg

SEQ ID NO: 5 - DNA, 32 bp, synthetic construct
  source 1..32: mol_type = other DNA; note = reverse primer R2 for amplification of dnaA
tgcatcacagttgtatgatcyykmccrccaaa

SEQ ID NO: 6 - DNA, 20 bp, synthetic construct
  source 1..20: mol_type = other DNA; note = forward primer F3 for amplification of rpoA
atgatygartttgaaaaacc

SEQ ID NO: 7 - DNA, 20 bp, synthetic construct
  source 1..20: mol_type = other DNA; note = reverse primer R3 for amplification of rpoA
achgtrttratdccdgcrcg

SEQ ID NO: 8 - DNA, 10 bp, synthetic construct
  source 1..10: mol_type = other DNA; note = primer RAPD-A for RAPD
aacgcgcaac

SEQ ID NO: 9 - DNA, 10 bp, synthetic construct
  source 1..10: mol_type = other DNA; note = primer RAPD-B for RAPD
gcggaaatag

SEQ ID NO: 10 - DNA, 10 bp, synthetic construct
  source 1..10: mol_type = other DNA; note = primer RAPD-C for RAPD
ctcaggtcgc

SEQ ID NO: 11 - DNA, 10 bp, synthetic construct
  source 1..10: mol_type = other DNA; note = primer RAPD-1 for RAPD
ccgcagccaa

(19) United States
(12) Patent Application Publication
(10) Pub. No.: US 2026/0130955 A1
(43) Pub. Date: May 14, 2026

(54) LEUCONOSTOC LACTIS LEL168 AND METHODS FOR INCREASING CALCIUM ABSORPTION AND INHIBITING BONE RESORPTION USING THE SAME

(71) Applicant: SYNBIO TECH INC., Kaohsiung City (TW)

(72) Inventors: Pei-Hsun TSAI, Kaohsiung City (TW); Yi-Chen CHENG, Kaohsiung City (TW); Chia-Chia LEE, Kaohsiung City (TW); Jin-Seng LIN, Tainan City (TW)

(21) Appl. No.: 19/291,835

(22) Filed: Aug. 6, 2025

Related U.S. Application Data
(63) Continuation of application No. PCT/CN2025/107052, filed on Jul. 4, 2025.
(60) Provisional application No. 63/718,156, filed on Nov. 8, 2024.

(51) Int. Cl. A61K 35/744; A61K 33/06; A61P 19/00; C12N 1/20; C12R 1/01

(52) U.S. Cl. CPC A61K 35/744; A61K 33/06; A61P 19/00; C12N 1/205; C12R 2001/01

(57) ABSTRACT

Leuconostoc lactis LEL168 is deposited at the Leibniz Institute DSMZ-German Collection of Microorganisms and Cell Cultures GmbH under an accession number DSM 35134 in accordance with the Budapest Treaty. Methods for increasing calcium absorption and inhibiting bone resorption using the Leuconostoc lactis LEL168, compositions for use in increasing calcium absorption and inhibiting bone resorption, uses of a composition in the manufacture of a medicament or a food product for increasing calcium absorption and inhibiting bone resorption in a subject, and pharmaceutical compositions for increasing calcium absorption and inhibiting bone resorption in a subject are also provided.

### CROSS-REFERENCE TO RELATED APPLICATION

This application is a continuation of International Application No. PCT/CN2025/107052, filed on Jul. 4, 2025, which claims priority to U.S. Provisional Patent Application No. 63/718,156, filed on Nov. 8, 2024. The aforesaid applications are incorporated by reference herein in their entirety.

### SEQUENCE LISTING XML

The Sequence Listing submitted concurrently herewith with a file name of “PE-71878-AM-SEQUENCE LISTING.xml,” a creation date of Jul. 22, 2025, and a size of 14,122 bytes, is part of the specification and is incorporated by reference in its entirety.

### FIELD

The present disclosure relates to Leuconostoc lactis LEL168 and methods for increasing calcium absorption and inhibiting bone resorption using the same.

### BACKGROUND

Calcium is an essential element that a human body needs in large amounts, and plays an important role in blood coagulation, muscle contraction, bone construction, and maintenance of normal heart rhythm and nerve function in the human body. However, when the human body is deficient in calcium, calcium deficiency-associated disorders such as osteoporosis may develop and bone resorption by osteoclasts may be stimulated, which results in bone loss and accelerating the development of osteoporosis.

A prevalent way for delaying or alleviating the calcium deficiency-associated disorders is to increase calcium intake by provision of calcium supplements, such as calcium carbonate, calcium oxide, calcium phosphate, and calcium lactate gluconate. Nevertheless, calcium contained in the calcium supplements may not be absorbed effectively by the intestine of a human body, while excessive use of the calcium supplements may increase urinary calcium level in a human body, resulting in formation of calcium-containing kidney stones.

In view of the aforesaid, there is still a need to develop an effective way for increasing calcium absorption and inhibiting bone resorption without causing undesirable side effects.

### SUMMARY

Therefore, in a first aspect, the present disclosure provides Leuconostoc lactis LEL168, which can alleviate at least one of the drawbacks of the prior art, and which is deposited at the Leibniz Institute DSMZ-German Collection of Microorganisms and Cell Cultures GmbH under an accession number DSM 35134 in accordance with the Budapest Treaty.

In a second aspect, the present disclosure provides a method for increasing calcium absorption, which can alleviate at least one of the drawbacks of the prior art, and which includes administering to a subject in need thereof a composition including a culture of Leuconostoc lactis LEL168, which is deposited at the Leibniz Institute DSMZ-German Collection of Microorganisms and Cell Cultures GmbH under an accession number DSM 35134 in accordance with the Budapest Treaty.

In a third aspect, the present disclosure provides a method for inhibiting bone resorption, which can alleviate at least one of the drawbacks of the prior art, and which includes administering to a subject in need thereof a composition including a culture of Leuconostoc lactis LEL168, which is deposited at the Leibniz Institute DSMZ-German Collection of Microorganisms and Cell Cultures GmbH under an accession number DSM 35134 in accordance with the Budapest Treaty.

In a fourth aspect, the present disclosure provides a composition for use in increasing calcium absorption, which can alleviate at least one of the drawbacks of the prior art. The use includes administering to a subject in need thereof a composition including a culture of Leuconostoc lactis LEL168, which is deposited at the Leibniz Institute DSMZ-German Collection of Microorganisms and Cell Cultures GmbH under an accession number DSM 35134 in accordance with the Budapest Treaty.

In a fifth aspect, the present disclosure provides a composition for use in inhibiting bone resorption, which can alleviate at least one of the drawbacks of the prior art. The use includes administering to a subject in need thereof a composition including a culture of Leuconostoc lactis LEL168, which is deposited at the Leibniz Institute DSMZ-German Collection of Microorganisms and Cell Cultures GmbH under an accession number DSM 35134 in accordance with the Budapest Treaty.

In a sixth aspect, the present disclosure provides use of a composition in the manufacture of a medicament or a food product for increasing calcium absorption in a subject, which can alleviate at least one of the drawbacks of the prior art. The composition includes a culture of Leuconostoc lactis LEL168, which is deposited at the Leibniz Institute DSMZ-German Collection of Microorganisms and Cell Cultures GmbH under an accession number DSM 35134 in accordance with the Budapest Treaty.

In a seventh aspect, the present disclosure provides use of a composition in the manufacture of a medicament or a food product for inhibiting bone resorption in a subject, which can alleviate at least one of the drawbacks of the prior art. The composition includes a culture of Leuconostoc lactis LEL168, which is deposited at the Leibniz Institute DSMZ-German Collection of Microorganisms and Cell Cultures GmbH under an accession number DSM 35134 in accordance with the Budapest Treaty.

In an eighth aspect, the present disclosure provides a pharmaceutical composition for increasing calcium absorption in a subject, which can alleviate at least one of the drawbacks of the prior art, and which includes a culture of Leuconostoc lactis LEL168, which is deposited at the Leibniz Institute DSMZ-German Collection of Microorganisms and Cell Cultures GmbH under an accession number DSM 35134 in accordance with the Budapest Treaty.

In a ninth aspect, the present disclosure provides a pharmaceutical composition for inhibiting bone resorption in a subject, which can alleviate at least one of the drawbacks of the prior art, and which includes a culture of Leuconostoc lactis LEL168, which is deposited at the Leibniz Institute DSMZ-German Collection of Microorganisms and Cell Cultures GmbH under an accession number DSM 35134 in accordance with the Budapest Treaty.

### BRIEF DESCRIPTION OF THE DRAWINGS

Other features and advantages of the disclosure will become apparent in the following detailed description of the embodiment(s) with reference to the accompanying drawings. It is noted that various features may not be drawn to scale.

FIG. 1 shows the relative fold change in calcium ion transport determined in each group of Example 2, infra.

FIG. 2 shows the relative fold change in TRAP activity determined in each group of Example 3, infra.

### DETAILED DESCRIPTION

For the purpose of this specification, it will be clearly understood that the word “comprising” means “including but not limited to”, and that the word “comprises” has a corresponding meaning.

It is to be understood that, if any prior art publication is referred to herein, such reference does not constitute an admission that the publication forms a part of the common general knowledge in the art.

Unless otherwise defined, all technical and scientific terms used herein have the meaning commonly understood by a person skilled in the art to which the present disclosure belongs. One skilled in the art will recognize many methods and materials similar or equivalent to those described herein, which could be used in the practice of the present disclosure. Indeed, the present disclosure is in no way limited to the methods and materials described.

In the development of various approaches to increase calcium absorption and inhibit bone resorption, the applicant has isolated a strain of Leuconostoc, which has been characterized as Leuconostoc lactis and named “Leuconostoc lactis LEL168” by the applicant, and which has been deposited at the Leibniz Institute DSMZ-German Collection of Microorganisms and Cell Cultures GmbH (Inhoffenstr. 7B, D-38124 Braunschweig, Germany) under an accession number DSM 35134 on Sep. 9, 2024 in accordance with the Budapest Treaty. The applicant surprisingly found that a culture of the Leuconostoc lactis LEL168 is capable of exhibiting excellent effects on improving intestinal calcium ion transport and suppressing osteoclast activity, and hence is expected to be useful for increasing calcium absorption and inhibiting bone resorption.

Therefore, the present disclosure provides Leuconostoc lactis LEL168, which is deposited at the Leibniz Institute DSMZ-German Collection of Microorganisms and Cell Cultures GmbH under an accession number DSM 35134 in accordance with the Budapest Treaty.

The present disclosure also provides a method for increasing calcium absorption, which includes administering to a subject in need thereof a composition including a culture of Leuconostoc lactis LEL168, which is deposited at the Leibniz Institute DSMZ-German Collection of Microorganisms and Cell Cultures GmbH under an accession number DSM 35134 in accordance with the Budapest Treaty.

The present disclosure also provides a method for inhibiting bone resorption, which includes administering to a subject in need thereof a composition including a culture of Leuconostoc lactis LEL168, which is deposited at the Leibniz Institute DSMZ-German Collection of Microorganisms and Cell Cultures GmbH under an accession number DSM 35134 in accordance with the Budapest Treaty.

As used herein, the term “administering” can be used interchangeably with other terms such as “administration”, and means introducing, providing or delivering a pre-determined active ingredient to a subject by any suitable routes to perform its intended function.

As used herein, the term “subject” refers to any animal of interest, such as humans, monkeys, cows, sheep, horses, pigs, goats, dogs, cats, mice, and rats.

According to the present disclosure, the subject suffers from a calcium deficiency-associated disorder. In certain embodiments, the calcium deficiency-associated disorder may be selected from the group consisting of osteoporosis, osteopenia, rickets, joint pain, muscle cramp, hypertension, palpitation, anxiety, obesity, skin inflammation, hypocalcemia, and combinations thereof. In certain embodiments, the subject suffers from osteoporosis. In other embodiments, the subject does not suffer from osteoporosis.

In certain embodiments, the subject may suffer from dysmenorrhea, or be in a perimenopausal, menopausal, or postmenopausal stage, resulting in a calcium deficiency.

According to the present disclosure, the culture of the Leuconostoc lactis LEL168 may be prepared by culturing the abovementioned Leuconostoc lactis LEL168 in a culture medium suitable for growth and/or proliferation thereof.

As used herein, the term “culturing” can be used interchangeably with other terms such as “fermentation” and “cultivation”.

According to the present disclosure, the culture medium suitable for culturing the Leuconostoc lactis LEL168 may be formulated using techniques well-known to those skilled in the art, or may be obtained as a commercial product which may include, but is not limited to, MRS (De Man, Rogosa and Sharpe) broth, MRS broth containing cysteine, and mineral medium containing glucose and soy peptone. In an exemplary embodiment, the culture medium is the MRS broth.

The procedures and parameter conditions for cultivation may be adjusted according to practical requirements. In this regard, those skilled in the art may refer to journal articles, e.g., Liu Z. et al. (2021), J Zhejiang Univ Sci B., 22(7):533-547.

According to the present disclosure, cultivation may be conducted at a temperature of 37° C. for a time period of 16 hours.

According to the present disclosure, the culture of the Leuconostoc lactis LEL168 may be a liquid culture.

According to the present disclosure, the liquid culture may have a total bacterial concentration ranging from 1×10⁶ CFU/mL to 1×10⁹ CFU/mL. In an exemplary embodiment, the liquid culture has a total bacterial concentration of 1×10⁷ CFU/mL.

According to the present disclosure, the liquid culture may be free of bacterial cells, which is obtained by subjecting a liquid culture formed after culturing the Leuconostoc lactis LEL168 to a solid-liquid separation treatment.

As used herein, the term “free of bacterial cells” means that the liquid culture lacks a significant amount of bacterial cells. In certain embodiments, the amount of the bacterial cells does not have a measurable effect on the properties of the liquid culture. In other embodiments, the liquid culture is completely free of bacterial cells.

According to the present disclosure, the solid-liquid separation treatment may be performed using techniques well-known to those skilled in the art. Examples of the solid-liquid separation treatment may include, but are not limited to, a centrifugation treatment, a filtration treatment, and a combination thereof. In an exemplary embodiment, the liquid culture is subjected to the centrifugation treatment and the filtration treatment in sequence so as to remove bacterial cells in the liquid culture, thereby obtaining a cell culture supernatant.

According to the present disclosure, the liquid culture may be free of viable bacterial cells, which is obtained by subjecting a liquid culture formed after culturing the Leuconostoc lactis LEL168 to a sterilization treatment.

According to the present disclosure, the culture may contain viable bacterial cells only, which is obtained by subjecting a liquid culture formed after culturing the Leuconostoc lactis LEL168 to the aforesaid solid-liquid separation treatment so as to remove a liquid portion from the liquid culture.

According to the present disclosure, the culture may be concentrated or non-concentrated, a liquid, a paste, a semi-solid, or a solid (e.g., a pellet, a granule, or a powder), and may be heat-inactivated, frozen, dried, freeze-dried, or spray/fluid bed-dried.

According to the present disclosure, the composition may further include a calcium supplement. Examples of the calcium supplement may include, but are not limited to, calcium chloride, calcium carbonate, calcium oxide, calcium dihydrogen phosphate, dicalcium phosphate, tricalcium bis(phosphate), calcium lactate gluconate, calcium L-threonate, calcium citrate, calcium lactate, and combinations thereof. In an exemplary embodiment, the calcium supplement is calcium chloride.

According to the present disclosure, the composition may be formulated as a food product using a standard technique well known to one of ordinary skill in the art. For example, the composition may be formulated in the form of a food additive, which is added to an edible material to prepare a food product for human or animal consumption.

As used herein, the term “food product” refers to any article or substance that can be ingested by a subject into the body thereof. Examples of the food product may include, but are not limited to, milk powders, fermented milk, yogurt, butter, beverages (e.g., tea, coffee, etc.), functional beverages, a flour product, baked foods, confectionery, candies, fermented foods, animal feeds, health foods, infant foods, and dietary supplements.

According to the present disclosure, the composition may be prepared in the form of a pharmaceutical composition. The pharmaceutical composition may be formulated into a dosage form suitable for oral administration using technology well known to those skilled in the art.

According to the present disclosure, examples of the dosage form suitable for oral administration may include, but are not limited to, sterile powders, tablets, troches, lozenges, pellets, capsules, dispersible powders or granules, solutions, suspensions, emulsions, syrup, elixir, slurry, and the like.

According to the present disclosure, the pharmaceutical composition may further include a pharmaceutically acceptable carrier widely employed in the art of drug-manufacturing. For instance, the pharmaceutically acceptable carrier may include one or more of the following agents: solvents, buffers, emulsifiers, suspending agents, decomposers, disintegrating agents, dispersing agents, binding agents, excipients, stabilizing agents, chelating agents, diluents, gelling agents, preservatives, wetting agents, lubricants, absorption delaying agents, liposomes, and the like. The choice and amount of the aforesaid agents are within the expertise and routine skills of those skilled in the art.

According to the present disclosure, the dose and frequency of administration of the culture of the Leuconostoc lactis LEL168 may vary depending on the following factors: the severity of the illness or disorder to be treated, routes of administration, and weight, age, physical condition and response of the subject to be treated. In general, the culture of the Leuconostoc lactis LEL168 may be administered orally in a single dose or in several doses.

The present disclosure also provides the aforesaid composition for use in increasing calcium absorption, which use includes administering to a subject in need thereof the aforesaid composition including the culture of the Leuconostoc lactis LEL168. The present disclosure also provides the aforesaid composition for use in inhibiting bone resorption, which use includes administering to a subject in need thereof the aforesaid composition including the culture of the Leuconostoc lactis LEL168.

The present disclosure further provides use of the aforesaid composition in the manufacture of a medicament or a food product for increasing calcium absorption in a subject. The present disclosure further provides use of the aforesaid composition in the manufacture of a medicament or a food product for inhibiting bone resorption in a subject.

The disclosure will be further described by way of the following examples. However, it should be understood that the following examples are solely intended for the purpose of illustration and should not be construed as limiting the disclosure in practice.

### EXAMPLES

### Example 1. Screening and Characterization of Isolated Strain of Leuconostoc lactis LEL168

### A. Source and Isolation of Isolate LEL168:

Feces from Paguma larvata were collected in Shoushan, Kaohsiung, Taiwan, China so as to serve as a sample source, followed by conducting isolation and screening of Leuconostoc using M17 agar bases (Manufacturer: HiMedia, Cat. no.: HM-M929), thereby obtaining an isolated strain of Leuconostoc spp., which is designated as isolate LEL168.

### B. Extraction of Genomic DNA:

The isolate LEL168 was subjected to extraction of genomic DNA with primarily reference to the method described in Zhu, H et al. (1993), Nucleic Acids Res., 21(22):5279-5280, in which an extraction buffer (containing 100 mM Tris and 40 mM EDTA; pH 9.0), Mini-BeadBeater Glass Mill Beads (0.1 mm) (Manufacturer: BioSpec Products, Cat. no. 11079101), and 500 μL of phenol-chloroform-isoamyl alcohol mixture (25:24:1, v/v/v) were used.

C. 16S Ribosomal DNA (rDNA) Sequence Analysis:

First, the resultant genomic DNA of the isolate LEL168 obtained in section B of this example, serving as a template, was subjected to polymerase chain reaction (PCR) using a designed primer pair specific for bacterial 16S rDNA gene shown in Table 1 and under the reaction conditions shown in Table 2 below, thereby obtaining a PCR product (i.e., PCR-amplified 16S rDNA fragments) of the isolate LEL168.

TABLE 1

| | Nucleotide sequence |
|---|---|
| Primer | (5′→3′) |
| Forward primer | agagtttgatcctggctcag |
| F1 | (SEQ ID NO: 1) |
| Reverse primer | ggttaccttgttacgact |
| R1 | (SEQ ID NO: 2) |

TABLE 2

| Reaction mix | Volume (μL) |
|---|---|
| Genomic DNA (10 ng/μL) | 1 |
| Forward primer F1 (10 μM) | 0.5 |
| Reverse primer R1 (10 μM) | 0.5 |
| Fast-Run ™ 2X Tag Master Mix | 10 |
| (Manufacturer: Protech, Cat. no.: SA-TMM228) | |
| Deionized water | 8 |

Operating conditions: initial denaturation at 95° C. for 5 minutes, followed by 30 cycles of denaturation at 95° C. for 60 seconds, primer annealing at 50° C. for 60 seconds, and extension at 72° C. for 60 seconds in sequence; and finally, elongation at 72° C. for 8 minutes.

After completing the PCR, the PCR product was subjected to 2% agarose gel electrophoresis to verify the presence of a PCR amplicon that is approximately 1500 bp in size, followed by subjecting the PCR amplicon to sequencing analysis, which was entrusted to Genomics BioSci & Tech Co., Ltd., thereby obtaining 16S rDNA sequence of the isolate LEL168 (SEQ ID No: 3). By comparing the data in the NCBI's gene database, the 16S rDNA sequence of the isolate LEL168 is found to have 100% identity to a part of the 16S rDNA sequence (NCBI accession no.: NR_040823.1) of Leuconostoc lactis KCTC3528 (DSM accession no.: 20202T, BCRC accession no.: 12261T).

In view of the aforesaid experimental results, the isolate LEL168 of the present disclosure is identified as Leuconostoc lactis. In order to confirm whether such Leuconostoc lactis LEL168 is a novel Leuconostoc lactis strain, the Leuconostoc lactis LEL168 was further subjected to the subsequent experiments as described in sections D and E of this example.

### D. Single Nucleotide Polymorphism (SNP) Analysis:

First, three known Leuconostoc lactis strains (i.e., BCRC 12261T, BCRC 14013, and BCRC 16060), which were obtained from the Bioresource Collection and Research Center (BCRC) of the Food Industry Research and Development Institute (FIRDI) (No. 331, Shih-Pin Rd., Hsinchu City 300, Taiwan, China), were subjected to extraction of genomic DNA using the aforesaid method described in section B of this example. Next, the resultant genomic DNA of such three known Leuconostoc lactis strains and the resultant genomic DNA of the isolate LEL168 obtained in section B of this example, serving as templates, were subjected to PCR using designed primer pairs specific for two bacterial housekeeping genes (i.e., dnaA and rpoA genes) shown in Table 3 and the reaction mixture shown in Table 4, and under the operating conditions shown in Table 5 below, thereby obtaining PCR products (i.e., PCR-amplified dnaA and rpoA fragments) of the aforesaid four Leuconostoc lactis isolates.

TABLE 3

| Target | | Nucleotide sequence |
|---|---|---|
| gene | Primer | (5′→3′) |
| dnaA | Forward primer | ggtggcgttggtctaggwa |
| | F2 | aaacmcayytratg |
| | | (SEQ ID NO: 4) |
| | Reverse primer | tgcatcacagttgtatgat |
| | R2 | cyykmccrccaaa |
| | | (SEQ ID NO: 5) |
| rpoA | Forward primer | atgatygartttgaaaaacc |
| | F3 | (SEQ ID NO: 6) |
| | Reverse primer | achgtrttratdccdgcrcg |
| | R3 | (SEQ ID NO: 7) |

TABLE 4

| Reaction mix | Volume (μL) |
|---|---|
| Genomic DNA (10 ng/μL) | 1 |
| Forward primer (10 μM) | 0.5 |
| Reverse primer (10 μM) | 0.5 |
| dNTPs (10 mM) | 0.5 |
| (Manufacturer: Protech, Cat. no.: PT-8010) | |
| 10× buffer solution | 2.5 |
| (Manufacturer: Protech, Cat. no.: PT-PROTAQ-B) | |
| ProTaq plus DNA polymerase (5 U/μL) | 0.5 |
| (Manufacturer: Protech, Cat. no.: PT-526) | |
| MgCl₂ (50 mM) | 1 |
| (Manufacturer: Protech, Cat. no.: PT-PROTAQ-B-1) | |
| Deionized water | 18.5 |

TABLE 5

| Primer pair | Operating conditions |
|---|---|
| Forward | Initial denaturation at 95° C. for 2 minutes, followed by 35 |
| primer F2/ | cycles of denaturation at 95° C. for 20 seconds, primer |
| Reverse | annealing at 55° C. for 30 seconds, and extension at 72° C. |
| primer R2 | for 75 seconds in sequence; and finally, elongation at 72° |
| | C. for 3 minutes. |
| Forward | Initial denaturation at 95° C. for 2 minutes, followed by 30 |
| primer F3/ | cycles of denaturation at 95° C. for 20 seconds, primer |
| Reverse | annealing at 54° C. for 30 seconds, and extension at 72° C. |
| primer R3 | for 75 seconds in sequence; and finally, elongation at 72° |
| | C. for 3 minutes. |

After completing the PCR, the PCR products were subjected to 2% agarose gel electrophoresis to verify the presence of a PCR amplicon that is approximately 800 bp in size, followed by subjecting the PCR amplicon to sequencing analysis, which was entrusted to Genomics BioSci & Tech Co., Ltd., thereby obtaining dnaA and rpoA gene sequences of the four Leuconostoc lactis isolates.

Thereafter, the dnaA and rpoA gene sequences of the four Leuconostoc lactis isolates were subjected to multiple sequence alignment analysis using Molecular Evolutionary Genetics Analysis (MEGA) software version 7.0 (Pennsylvania State University, USA), so as to determine and identify single nucleotide polymorphisms (SNPs) in the dnaA and rpoA genes. Subsequently, genotyping of the four Leuconostoc lactis isolates was conducted by analyzing the genetic variation of the SNPs within the dnaA and rpoA genes of these Leuconostoc lactis isolates. The experimental results are shown in Table 8 below.

### E. Random Amplified Polymorphic DNA (RAPD) Analysis:

The RAPD analysis was generally performed with reference to Akopyanz N. et al. (1992), Nucleic Acids Res., 20:5137-5142. Briefly, the resultant genomic DNA of the isolate LEL168 obtained in section B of this example and the resultant genomic DNA of the three known Leuconostoc lactis strains obtained in section D of this example, serving as templates, were subjected to PCR using four primers (i.e., RAPD-A, RAPD-B, RAPD-C, and RAPD-1) shown in Table 6 and under the reaction conditions shown in Table 7 below.

TABLE 6

| | Nucleotide sequence |
|---|---|
| Primer | (5′→3′) |
| RAPD-A | aacgcgcaac (SEQ ID NO: 8) |
| RAPD-B | goggaaatag (SEQ ID NO: 9) |
| RAPD-C | ctcaggtcgc (SEQ ID NO: 10) |
| RAPD-1 | ccgcagccaa (SEQ ID NO: 11) |

TABLE 7

| Reaction mix | Volume (μL) |
|---|---|
| Genomic DNA (10 ng/μL) | 1 |
| Primer (10 μM) | 4 |
| 2× Tag Master Mix | 12.5 |
| (Manufacturer: Ampliqon, Cat. no.: 5200300) | |
| MgCl₂ (25 mM) | 1.5 |
| (Manufacturer: Sigma, Cat. no.: M8787) | |
| Deionized water | 6 |

Operating conditions: initial denaturation at 94° C. for 2 minutes, followed by 6 cycles of denaturation at 94° C. for 30 seconds, primer annealing at 36° C. for 60 seconds, and extension at 72° C. for 90 seconds in sequence; and then by 30 cycles of denaturation at 94° C. for 20 seconds, primer annealing at 36° C. for 30 seconds, and extension at 72° C. for 90 seconds in sequence; and finally, elongation at 72° C. for 3 minutes.

After completing the PCR, the resultant PCR products were subjected to 2% agarose gel electrophoresis, followed by staining and then visualization under ultraviolet light for photography, thereby obtaining genetic fingerprint profiles of the four Leuconostoc lactis isolates. These genetic fingerprint profiles were then analyzed and compared to conduct genotyping of these Leuconostoc lactis isolates.

The experimental results from sections D and E of this example are summarized in Table 8 below. As shown in Table 8, by virtue of combining these two genotyping methods (i.e., the SNP and RAPD analyses), the four Leuconostoc lactis isolates were identified as genetically distinct strains.

TABLE 8

| | | | |
|---|---|---|---|
| Leuconostoc lactis | SNP analysis | RAPD | Combined |

| isolate | dnaA | rpoA | analysis | results |
|---|---|---|---|---|
| LEL168 | I | i | a | 1 |
| BCRC 12261^{T} | II | ii | b | 2 |
| BCRC 14013 | III | i | c | 3 |
| BCRC 16060 | III | iii | d | 4 |

Note 1:
Each distinct letter or symbol in a column represents a different genotype as determined by a same genotyping method.
Note 2:
SNP analysis of the dnaA and rpoA genes identified at least 10 and 5 SNP sites, respectively.

Based on the aforementioned characterization results, the applicant concludes that the Leuconostoc lactis LEL168 of the present disclosure is a novel Leuconostoc lactis strain. As such, the Leuconostoc lactis LEL168 has been deposited at the Bioresource Collection and Research Center (BCRC) of the Food Industry Research and Development Institute (FIRDI) (No. 331, Shih-Pin Rd., Hsinchu City 300, Taiwan, China) under an accession number BCRC 911240 on Aug. 28, 2024, and has also been deposited at the Leibniz Institute DSMZ-German Collection of Microorganisms and Cell Cultures GmbH (Inhoffenstr. 7B, D-38124 Braunschweig, Germany) under an accession number DSM 35134 on Sep. 9, 2024 in accordance with the Budapest Treaty.

### Example 2. Evaluation of the Efficacy of Leuconostoc lactis LEL168 in Increasing Intestinal Calcium Ion Absorption

In this example, the evaluation of calcium absorption efficacy was conducted with primarily reference to the method described in Raveschot, C. et al., (2020), Food research international (Ottawa, Ont.), 133:109201.

### Experimental Materials:

### 1. Source and Cultivation of Human Colon Adenocarcinoma Cell Line Caco-2

Human colon adenocarcinoma cell line Caco-2 (ATCC® HTB-37™) used in this example was obtained from the BCRC of the FIRDI. The Caco-2 cells were grown in a 10-cm Petri dish containing Dulbecco's Modified Eagle's Medium (DMEM, Manufacturer: Gibco, Cat. no.: 10569-010) supplemented with 10% fetal bovine serum (FBS, Manufacturer: Gibco, Cat. no.: A52567) and 1% penicillin-streptomycin-amphotericin B (Manufacturer: Biological Industries, Cat. no.: 03-033-1B), and were cultivated in an incubator at 37° C. with 5% CO₂. Subsequently, medium change was performed every two to three days. Cell passage was performed when the cultured cells reached 80% to 90% of confluence.

2. Preparation of Bacterial Suspension of Leuconostoc lactis LEL168

First, the Leuconostoc lactis LEL168 obtained from section A of Example 1 was inoculated in a MRS broth, followed by cultivation in an incubator at 37° C. for 16 hours. Next, such inoculation and cultivation procedures were repeated twice to activate the Leuconostoc lactis LEL168. Subsequently, the resultant culture was subjected to centrifugation at 5000 rpm and 4° C. for 5 minutes, followed by discarding the resultant supernatant, so as to collect the resultant bacterial pellet. Thereafter, the resultant bacterial pellet was washed three times with phosphate-buffered saline (PBS). Afterwards, an appropriate amount of PBS was added to suspend the washed cell pellet and to adjust the bacterial concentration to the desired bacterial concentration, so as to obtain a bacterial suspension of the Leuconostoc lactis LEL168, which had a bacterial concentration of 10⁹ CFU/mL (abbreviated as LEL168-bacterial suspension).

### Experimental Procedures:

First, the Caco-2 cells prepared in section 1 of “Experimental Materials” of this example were divided into 2 groups, including a control group and an experimental group. Each group of the Caco-2 cells was seeded at a concentration of 4×10⁴ cells/well into a respective Millicell® hanging cell culture insert (Manufacturer: Millipore, Cat. no.: PTHT24H48, having a polyethylene terephthalate (PET) membrane with a pore size of 0.4 μm) containing 400 μL of DMEM supplemented with 10% FBS and 1% penicillin-streptomycin-amphotericin B. Next, the respective Millicell® hanging cell culture insert was placed into each well of a 24-well plate containing 700 μL of DMEM, followed by cultivation in an incubator (37° C., 5% CO₂) for 21 days, and medium change was performed every two to three days, so that the Caco-2 cells formed a cell monolayer on a bottom of the respective Millicell® hanging cell culture insert. Afterwards, the liquid in the respective Millicell® hanging cell culture insert was replaced with 300 μL of DMEM supplemented with 2% FBS and a treating agent(s) (i.e., calcium chloride (Manufacturer: Sigma, Cat. no.: C7902) and/or the LEL168-bacterial suspension prepared in section 2 of “Experimental Materials” of this example) with a specific final concentration as shown in Table 9 below. In addition, the liquid in each well of the 24-well plate was replaced with 600 μL of fresh DMEM supplemented with 2% FBS, followed by cultivation in an incubator (37° C., 5% CO₂) for 24 hours. Subsequently, the liquid in each well of the 24-well plate was collected, and was then subjected to determination of calcium ion concentration using a Calcium Colorimetric Assay Kit (Manufacturer: Sigma, Cat. no.: MAK022).

TABLE 9

Treating agent with final concentration

| | | LEL168-bacterial |
|---|---|---|
| Group | Calcium chloride | suspension |
| Control group | 4 mM | — |
| Experimental group | 4 mM | 1 × 10⁷ CFU/mL |

The relative fold change in calcium ion transport was calculated by substituting the determined calcium ion concentration into the following Equation (1):

$$A = B / C \qquad ( 1 )$$

where A=relative fold change in calcium ion transport
   B=calcium ion concentration determined in each group
   C=calcium ion concentration determined in the control group

### Results:

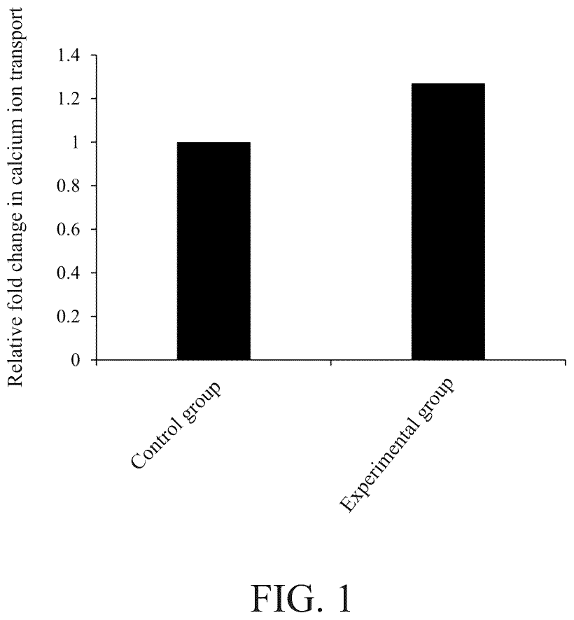

FIG. 1 shows the relative fold change in calcium ion transport determined in each group. As shown in FIG. 1, compared with the control group, the relative fold change in calcium ion transport determined in the experimental group showed a substantial increase. This result demonstrates that the bacterial suspension of the Leuconostoc lactis LEL168 can effectively improve intestinal calcium ion transport, and hence the culture of the Leuconostoc lactis LEL168 of the present disclosure is expected to be useful for increasing calcium absorption.

### Example 3. Evaluation of the Efficacy of Leuconostoc lactis LEL168 in Inhibiting Bone Resorption

In this example, the evaluation of bone resorption efficacy was conducted with primarily reference to the method described in Quach D. et al., (2019), Bone Rep., 11:100227.

### Experimental Materials:

### 1. Source and Cultivation of Mouse Macrophage Cell Line RAW264.7

Mouse macrophage cell line RAW264.7 used in this example was obtained from the BCRC of the FIRDI. The RAW264.7 cells were grown in a 10-cm Petri dish containing DMEM supplemented with 10% FBS and 1% penicillin-streptomycin-amphotericin B (Manufacturer: Biological Industries, Cat. no.: 03-033-1B), and were cultivated in an incubator at 37° C. with 5% CO₂. Subsequently, medium change was performed every two to three days. Cell passage was performed when the cultured cells reached 80% to 90% of confluence.

2. Preparation of Culture Supernatant of Leuconostoc lactis LEL168

The LEL168-bacterial suspension prepared in section 2 of “Experimental Materials” of Example 2 above was subjected to centrifugation at 5000 rpm for 5 minutes, followed by discarding the resultant supernatant, so as to collect the resultant bacterial pellet. Subsequently, the resultant bacterial pellet was re-suspended in 6 mL of Minimum Essential Medium (MEM, Manufacturer: Sigma, Cat. no.: M2279) supplemented with 10% FBS, 1% non-essential amino acids (NEAA, Manufacturer: Sigma, Cat. no.: M7145), and 1% L-glutamine (Manufacturer: Sigma, Cat. no.: G7513), followed by placing the resultant suspension in an shaking incubator with temperature control (37° C., 100 rpm) for cultivation for 24 hours. Thereafter, the resultant culture was subjected to centrifugation at 5000 rpm for 5 minutes, and the resultant culture supernatant was then collected. Afterwards, a pH value of the resultant culture supernatant was adjusted to between 7.0 and 7.6 using a 4N sodium hydroxide solution, followed by performing a filtration treatment with a filter having a pore size of 0.22 μm. The filtrate thus obtained served as a culture supernatant of the Leuconostoc lactis LEL168 (abbreviated as LEL168-culture supernatant) to be used in the following experiments.

### Experimental Procedures:

### A. Induction of Macrophage-to-Osteoclast Differentiation

First, the RAW264.7 cells prepared in section 1 of “Experimental Materials” of this example were divided into 3 groups, including a normal control group, a pathological control group, and an experimental group. Each group of the RAW264.7 cells was seeded at a concentration of 1.2×10⁴ cells/well into each well of a 24-well plate containing 800 μL of DMEM supplemented with 10% FBS and 1% penicillin-streptomycin-amphotericin B, followed by cultivation in an incubator (37° C., 5% CO₂) for 24 hours. Afterwards, the liquid in each well of the 24-well plate was replaced with 800 μL of MEM supplemented with 10% FBS, 1% NEAA, 1% L-glutamine, 1% penicillin-streptomycin-amphotericin B, and/or a treating agent(s) (i.e., receptor activator of NF-κB ligand (RANKL, Manufacturer: abcam, Cat. no.: ab129136) and/or the LEL168-culture supernatant prepared in section 2 of “Experimental Materials” of this example) with a specific final concentration as shown in Table 10 below, followed by cultivation in an incubator (37° C., 5% CO₂) for 7 days. During cultivation, change of medium supplemented with the treating agent(s) was performed every two to three days.

TABLE 10

| | Treating agent with final concentration | |
|---|---|---|
| | | LEL168-culture |
| Group | RANKL | supernatant |
| Normal control group | — | — |
| Pathological control group | 100 ng/mL | — |
| Experimental group | 100 ng/mL | 7.5 vol % |

Note:
The RANKL was used to induce differentiation of macrophage into osteoclasts.

### B. Analysis of Tartrate-Resistant Acid Phosphatase (TRAP) Activity

After cultivation, the resultant liquid in each well obtained in section A of this example was removed, and each well was washed with phosphate-buffered saline (PBS). Subsequently, the cell culture in each well of the 24-well plate was added with 200 μL of CelLytic™ M Cell Lysis Reagent (Manufacturer: Sigma, Cat. no.: C3228), and the 24-well plate was then incubated with shaking at room temperature for a time period ranging from 10 minutes to 15 minutes to lysis the RAW264.7 cells. Thereafter, the resultant cell lysate was subjected to centrifugation at 13000 at 4° C. for 5 minutes, followed by collecting the resultant supernatant. Then, the resultant supernatant of each group was subjected to determination of TRAP activity and total protein concentration using an Acid Phosphatase Assay Kit (Manufacturer: abcam, Cat. no.: ab83367) and a Protein Assay Dye Reagent Concentrate (Manufacturer: Bio-Rad Laboratories, Inc., Cat. no.: 5000006), respectively. Afterwards, the determined TRAP activity of each group was normalized with the determined total protein concentration of the same group, and the relative fold change in TRAP activity for each group was further calculated by substituting the thus normalized TRAP activity into the following Equation (2):

$$D = E / F \qquad ( 2 )$$

where D=relative fold change in TRAP activity
   E=normalized TRAP activity of each group
   F=normalized TRAP activity of the pathological control group

### Results:

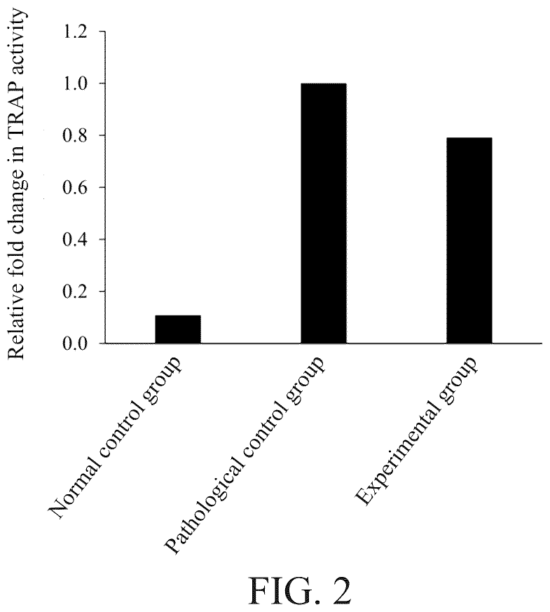

FIG. 2 shows the relative fold change in TRAP activity determined in each group. As shown in FIG. 2, compared with the normal control group, the relative fold change in TRAP activity determined in the pathological control group showed a substantial increase, indicating that the RAW264.7 cells had differentiated into highly active osteoclasts. In contrast, compared with the pathological control group, the relative fold change in TRAP activity determined in the experimental group showed a substantial decrease, demonstrating that the culture supernatant of the Leuconostoc lactis LEL168 was capable of effectively suppressing osteoclast activity. Therefore, the culture of the Leuconostoc lactis LEL168 of the present disclosure is expected to be useful for inhibiting bone resorption.

Summarizing the above test results, it is clear that the culture of the Leuconostoc lactis LEL168 (including the bacterial cells portion and the culture supernatant without (viable) bacterial cells) is capable of effectively increasing calcium absorption and inhibiting bone resorption.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, the one or more features may be singled out and practiced alone without the another one or more features or specific details. It should be further noted that one or more features or specific details from one embodiment may be practiced together with one or more features or specific details from another embodiment, where appropriate, in the practice of the disclosure.

While the disclosure has been described in connection with what is (are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A method for increasing calcium absorption, comprising administering to a subject in need thereof a composition including a culture of Leuconostoc lactis LEL168, which is deposited at the Leibniz Institute DSMZ-German Collection of Microorganisms and Cell Cultures GmbH under an accession number DSM 35134 in accordance with the Budapest Treaty.

2. The method as claimed in claim 1, wherein the culture is a liquid culture.

3. The method as claimed in claim 2, wherein the liquid culture is free of bacterial cells.

4. The method as claimed in claim 1, wherein the culture contains bacterial cells only.

5. The method as claimed in claim 1, wherein the subject suffers from a calcium deficiency-associated disorder.

6. The method as claimed in claim 1, wherein the composition further includes a calcium supplement.

7. The method as claimed in claim 1, wherein the composition is a food product.

8. The method as claimed in claim 1, wherein the composition is a pharmaceutical composition.

9. The method as claimed in claim 8, wherein the pharmaceutical composition is in a dosage form for oral administration.

10. A method for inhibiting bone resorption, comprising administering to a subject in need thereof a composition including a culture of Leuconostoc lactis LEL168, which is deposited at the Leibniz Institute DSMZ-German Collection of Microorganisms and Cell Cultures GmbH under an accession number DSM 35134 in accordance with the Budapest Treaty.

11. The method as claimed in claim 10, wherein the culture is a liquid culture.

12. The method as claimed in claim 11, wherein the liquid culture is free of bacterial cells.

13. The method as claimed in claim 10, wherein the culture contains bacterial cells only.

14. The method as claimed in claim 10, wherein the subject suffers from a calcium deficiency-associated disorder.

15. The method as claimed in claim 10, wherein the composition further includes a calcium supplement.

16. The method as claimed in claim 10, wherein the composition is a food product.

17. The method as claimed in claim 10, wherein the composition is a pharmaceutical composition.

18. The method as claimed in claim 17, wherein the pharmaceutical composition is in a dosage form for oral administration.
